(19) United States
(12) Patent Application Publication
(10) Pub. No.: US 2022/0218866 A1
(43) Pub. Date: Jul. 14, 2022

(54) A Sanitizing Apparatus for Sanitizing a Fluid and a Sanitizing Method Thereof

(71) Applicant: Champs Innovations Pte. Ltd., Singapore (SG)

(72) Inventors: Ing Jen Cheong, Singapore (SG); Wei Sheng Lance Tan, Singapore (SG); Krishna Kumar Manippady, Singapore (SG)

(21) Appl. No.: 17/612,975

(22) PCT Filed: May 20, 2020
(86) PCT No.: PCT/SG20/50300

§ 371 Date: Nov. 19, 2021

(30) Foreign Application Priority Data
May 22, 2019 (SG) 10201904566W

(51) Int. Cl. A61L 9/20; C02F 1/32

(52) U.S. Cl. CPC A61L 9/20; C02F 1/325; A61L 2209/12; F24F 8/22; C02F 2303/04; C02F 2201/3222; C02F 2201/328; A61L 2209/131

(57) ABSTRACT

A sanitizing apparatus for sanitizing a fluid is provided. The sanitizing apparatus includes a chamber having an inlet for receiving the fluid thereinto and an outlet for discharging the fluid therefrom, a divider adapted to divide the chamber into an upstream portion in fluid communication with the inlet and a downstream portion in fluid communication with the outlet, a sanitizing light source disposed in the upstream portion and adapted to emit a sanitizing light to sanitize the fluid therein, such that the divider is adapted to obstruct the fluid flow from the inlet to increase the density of the fluid in the upstream portion wherein the fluid is sanitized before being discharged from the chamber via the outlet. A sanitizing method for sanitizing a fluid is provided.

### CROSS-REFERENCE TO RELATED APPLICATIONS

The present application claims the benefit of Singapore Application No. 10201904566W filed 22 May 2019, which is incorporated by reference herein.

### TECHNICAL FIELD

The present invention relates to a sanitizing apparatus for sanitizing a fluid and a method thereof.

### BACKGROUND

The air quality of outdoor environment varies and depends on factors such as emissions from industries and traffic exhaust. However, in indoors, the air can be two to five times more polluted than the outdoor air. As the humidity indoor can be relatively higher than outdoor, the air particles with moisture increase the breeding and becomes a transmitting ground for harmful biological matter, such as viruses, bacteria, mould, and yeasts.

It is known that ultraviolet (“UV”) light sterilizes DNA so that the harmful biological matter exposed to UV light either dies or ceases reproduction. This property of a standard UV light has been utilized to sterilize air in a confined space. Commercially available UV source usually comes in the form of UV mercury lamps. However, a major drawback is that such a UV light source is harsh on the environment and harmful to human being. Air filters tend to be bulky due to the size of mercury lamps and are not suitable for portable use.

It has also been known that chemical-based UV sanitizer are used for air disinfection. However, such sanitizer, e.g. chemical aerosols or perfumes, do more harm than good. There are other types of air cleansing devices but each have one or more disadvantages. HEPA filters and cyclonic separation machines have been used to remove particles, but not for disinfection sanitization as filters can only remove particles not the bacteria. Other types of UV sanitizer have inefficient use of UV source. Light emitting devices (LED) are a recent invention which provide the ideal non-mercury-based UV light source. However, UV LED are still not an economically viable technology and are limited by low exposure reach. For example, the UV LED have limited active region of disinfection around the UV source. Due to the lack of UV exposure and residence time, it does not effectively kill microbes.

UV C light for air disinfection is not widely used as it is ineffective due to insufficient UV exposure. This is because microbes must be exposed to UV C for a certain period of time to render it inactive.

Hence, a solution is required to solve the abovementioned problem while achieving the objective of sanitizing air or liquid.

### SUMMARY

According to various embodiments, a sanitizing apparatus for sanitizing a fluid is provided. The sanitizing apparatus includes a chamber having an inlet for receiving the fluid thereinto and an outlet for discharging the fluid therefrom, a divider adapted to divide the chamber into an upstream portion in fluid communication with the inlet and a downstream portion in fluid communication with the outlet, a sanitizing light source disposed in the upstream portion and adapted to emit a sanitizing light to sanitize the fluid therein, such that the divider is adapted to obstruct the fluid flow from the inlet to increase the density of the fluid in the upstream portion wherein the fluid is sanitized before being discharged from the chamber via the outlet.

According to various embodiments, the divider may be adapted to retain the fluid from the inlet in the upstream portion to be sanitized and release the fluid into the downstream portion to be discharged from the chamber via the outlet.

According to various embodiments, the divider may include at least one opening adapted to toggle between a closed configuration to prevent the fluid from passing through and an opened configuration to allow the fluid to pass therethrough.

According to various embodiments, the divider may include at least one opening and may be flexible, such that the divider may be adapted to flex between an original position and a flexed position, such that in the original position, the at least one opening may be closed to prevent the fluid therethrough and in the flexed position, the at least one opening may be opened to allow the fluid therethrough.

According to various embodiments, the divider may be adapted to be flexed by the fluid in the upstream chamber from the original position to the flexed position, such that in the flexed position, the at least one opening may be opened for the fluid to be released from the upstream chamber.

According to various embodiments, may further include an actuator adapted to actuate the divider between the original position and the flexed position, such that in the flexed position, the at least one opening may be opened for the fluid to be released from the upstream chamber.

According to various embodiments, the divider may include a plurality of micropores adapted to allow the fluid therethrough.

According to various embodiments, the divider may include a fibre optic mesh comprising the plurality of micropores and adapted to transmit and emit the sanitizing light onto the fluid therethrough.

According to various embodiments, the sanitizing apparatus may further include a restrictor adapted to restrict the fluid flow from the inlet therethrough.

According to various embodiments, the restrictor may include a fibre optic mesh including a plurality of micro openings and adapted to transmit and emit the sanitizing light onto the fluid therethrough.

According to various embodiments, the divider may include a rough surface facing the inlet, such that the rough surface may be adapted to reduce the fluid flow rate along the divider.

According to various embodiments, the sanitizing apparatus may further include an inlet pump adapted to pump fluid into the chamber via the inlet.

According to various embodiments, the sanitizing apparatus may further include an outlet pump adapted to draw fluid out of the chamber via the outlet.

According to various embodiments, a sanitizing method for sanitizing a fluid is provided. The method includes receiving the fluid into an upstream portion of a divider of a chamber via an inlet, obstructing the fluid flow from the inlet by the divider to increase the density of the fluid in the upstream portion, sanitizing the fluid in the upstream portion before discharging the fluid from the chamber via the outlet.

According to various embodiments, the method may further include retaining the fluid from the inlet in the upstream portion to be sanitized and releasing the fluid into a downstream portion of the divider to be discharged from the chamber via the outlet.

According to various embodiments, the method may further include toggling at least one opening between a closed configuration to prevent the fluid from passing through and an opened configuration to allow the fluid to pass therethrough.

According to various embodiments, the method may include flexing the divider between an original position and a flexed position, such that in the original position, at least one opening of the divider may be closed to prevent the fluid therethrough and in the flexed position, the at least one opening may be opened to allow the fluid therethrough.

According to various embodiments, the divider may be adapted to be flexed by the fluid in the upstream chamber from the original position to the flexed position, such that in the flexed position, the at least one opening may be opened for the fluid to be released from the upstream chamber.

According to various embodiments, the method may further include actuating the divider between the original position and the flexed position, such that in the flexed position, the at least one opening may be opened for the fluid to be released from the upstream chamber.

According to various embodiments, the method may further include pumping the fluid into chamber via the inlet.

According to various embodiments, the method may further include drawing the fluid out of the chamber via the outlet.

### BRIEF DESCRIPTION OF DRAWINGS

FIG. 1 shows a schematic diagram of an exemplary embodiment of a sanitizing apparatus for sanitizing a fluid.

FIG. 2 shows a schematic diagram of an exemplary embodiment of the sanitizing apparatus.

FIG. 3A and FIG. 3B show schematic diagrams of an exemplary embodiment of the sanitizing apparatus.

FIG. 4A-FIG. 4D show schematic diagrams of different stages of an exemplary embodiment of the sanitizing apparatus.

FIG. 5 shows a schematic diagram of an exemplary embodiment of the sanitizing apparatus.

FIG. 6A-FIG. 6C show schematic diagrams of different stages of an exemplary embodiment of the sanitizing apparatus.

FIG. 7 shows a schematic diagram of an exemplary embodiment of the sanitizing apparatus.

FIG. 8 shows a schematic diagram of an exemplary embodiment of the sanitizing apparatus.

FIG. 9A and FIG. 9B show top and elevation views of an exemplary embodiment of the divider as shown in one of the embodiments in FIG. 4-FIG. 6.

FIG. 10 shows a schematic diagram of deflectors used in the sanitizing apparatus.

FIG. 11 shows an exemplary sanitizing method for sanitizing a fluid.

### DETAILED DESCRIPTION

In the following examples, reference will be made to the figures, in which identical features are designated with like numerals.

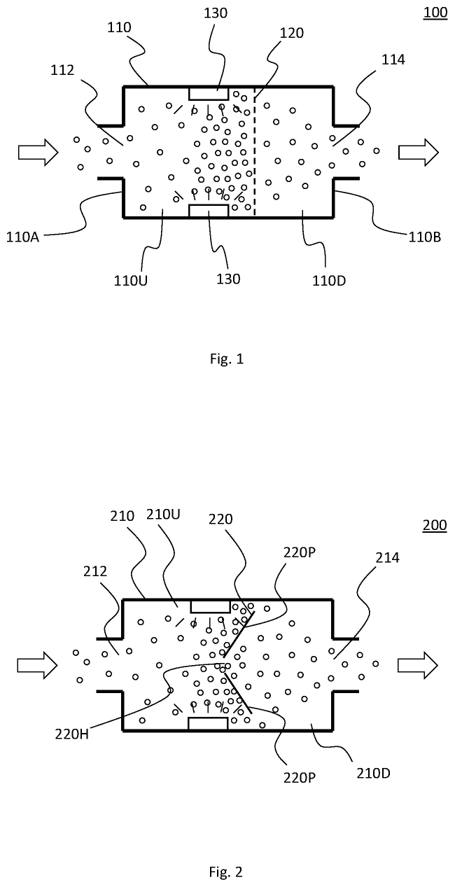

FIG. 1 shows a schematic diagram of an exemplary embodiment of a sanitizing apparatus 100 for sanitizing a fluid. Sanitizing apparatus 100 includes a chamber 110 having an inlet 112 for receiving the fluid thereinto and an outlet 114 for discharging the fluid therefrom, a divider 120 adapted to divide the chamber 110 into an upstream portion 110U in fluid communication with the inlet 112 and a downstream portion 110D in fluid communication with the outlet 114, a sanitizing light source 130 disposed in the upstream portion 110U and adapted to emit a sanitizing light to sanitize the fluid therein, such that the divider 120 is adapted to obstruct the fluid flow from the inlet 112 to increase the density of the fluid in the upstream portion 110U wherein the fluid is sanitized before being discharged from the chamber 110 via the outlet 114.

Chamber 110 may be tubular and extends from a proximal end 110A to a distal end 110B opposite the proximal end 110A. Inlet 112 may be disposed at the proximal end 110A and the outlet 114 may be disposed at the distal end 110B. Divider 120 may be disposed between the inlet 112 and the outlet 114 and extend across the chamber 110. Divider 120 may be disposed at the outlet 114 such that upon flowing through the divider 120, the fluid may be discharged from the chamber 110 via the outlet 114. Fluid may include gas and/or liquid. The interior surface of the chamber 110 may be reflective to reflect the sanitizing light from the sanitizing light source 130 onto the fluid so that exposure of the fluid to the sanitizing light is maximised.

The fluid may be directed into the chamber 110 via the inlet 112 and flow from the inlet 112 towards the divider 120. Fluid may be pumped into the chamber 110 such that the fluid is being pressured into the chamber. As the fluid flow is obstructed by the divider 120, the fluid may be compressed within the upstream portion 110U of the chamber 110. Hence, the fluid pressure within the upstream portion 110U may be higher than the downstream portion 110D of the chamber 110. As the fluid is being agglomerated and compressed, the density of the fluid increases. At the same time, the fluid is being sanitized by the sanitizing light emitted from the sanitizing light source 130. By obstructing the fluid flow and increasing the density of the fluid in the upstream portion 110U, the exposure time of the fluid to the sanitizing light is increased and the amount of fluid being sanitized per unit is increased.

To enable the sanitizing light source 130 to be effective, localised compacting of the biological matter loaded fluid molecules is beneficial. This provides improved residence exposure time for effective fluid sanitization. Sanitizing apparatus 100 utilizes the sanitizing light source 130 to disinfect the fluid that is being locally concentrated and compacted in the upstream portion 110U of the chamber 110. Fluid molecule in the upstream portion 110U may also be randomised and agglomerated. As shown in FIG. 1, the compaction may be achieved by using the divider 120 in the chamber 110. With a simple configuration, the sanitizing apparatus 100 may be small and portable such that it is suitable for a table-top air sanitizer. Sanitizing apparatus 100 may be used indoors, in confined space and also as a portable sanitizer.

FIG. 2 shows a schematic diagram of an exemplary embodiment of the sanitizing apparatus 200. Sanitizing apparatus 200 has similar features as the sanitizing apparatus 100 in FIG. 1. The same features have the same last two digits and letter (if any) in their reference numbers. Divider 220 may include a pair of panels 220P spaced apart from each other and extending laterally across the chamber 210 such that the pair of panels 220P forms a through hole 220H therebetween. Pair of panels 220P may be spaced from the internal wall of the chamber 210 to allow fluid flow between the chamber 210 wall and the pair of panels 220P. Through hole 220H may be small enough to allow a relatively small amount of fluid through to achieve the compression effect. Fluid from the inlet 212 may flow towards the divider 220, which is adapted to obstruct the fluid flow to the outlet 214. As shown in FIG. 2, a portion of the fluid may be accumulated and compressed in the upstream portion 210U of the chamber 210, while another portion may flow through the through hole 220H into the downstream portion 210D of the chamber 210. Divider 220 may include a rough surface facing the inlet 222, such that the rough surface may be adapted to reduce the fluid flow rate along the divider 220. As the fluid density increases in the upstream portion 210U, the fluid is being sanitized by the sanitizing light. Pair of panels 220P may be arranged in a V-shaped configuration where the apex of the V-shape is facing the inlet 212. Divider 220 may include a frustoconical cone with its tip facing the inlet 212.

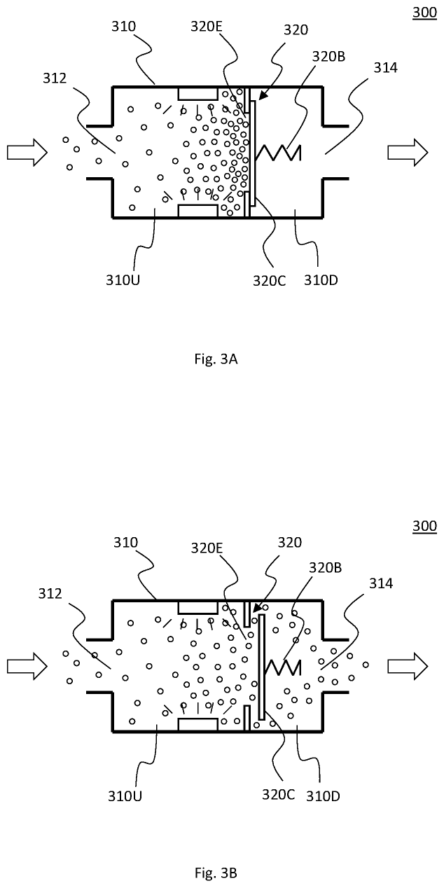

FIG. 3A and FIG. 3B show schematic diagrams of an exemplary embodiment of the sanitizing apparatus 300. Sanitizing apparatus 300 has similar features as the sanitizing apparatus 100 in FIG. 1. The same features have the same last two digits and letter (if any) in their reference numbers. Divider 320 may be adapted to retain the fluid from the inlet 312 in the upstream portion 310U to be sanitized as shown in FIG. 3A and release the fluid into the downstream portion 310D to be discharged from the chamber 310 via the outlet 314 as shown in FIG. 3B. Referring to FIG. 3A, the divider 320 may include at least one opening 320E adapted to toggle between a closed configuration (as shown in FIG. 3A) to prevent the fluid from passing through and an opened configuration (as shown in FIG. 3B) to allow the fluid to pass therethrough. Divider 320 may include a biasing cap 320C adapted to cover the opening 320E. Biasing cap 320C may be biased against the opening 320E such that the opening 320E is covered and sealed by the biasing cap 320C to prevent fluid through the opening 320E, i.e. closed configuration. Biasing cap 320C may be biased by a biasing member 320B, e.g. a spring, against the opening 320E. As the fluid is being compacted in the upstream portion 310U of the chamber 310, the fluid may be sanitized by the sanitizing light. Referring to FIG. 3B, as the fluid may exert a pressure onto the divider 320, when the pressure is sufficient to overcome the biasing force exerted by the biasing member 320B, the biasing cap 320C may uncover the opening 320E, i.e. opened configuration, and release the fluid through the opening 320E from the upstream portion 310U to the downstream portion 310D of the chamber 310. When the pressure is reduced after the fluid is released and is less than the biasing force, the biasing cap 320C may be pushed back to the closed configuration by the biasing member 320B to close the opening 320E.

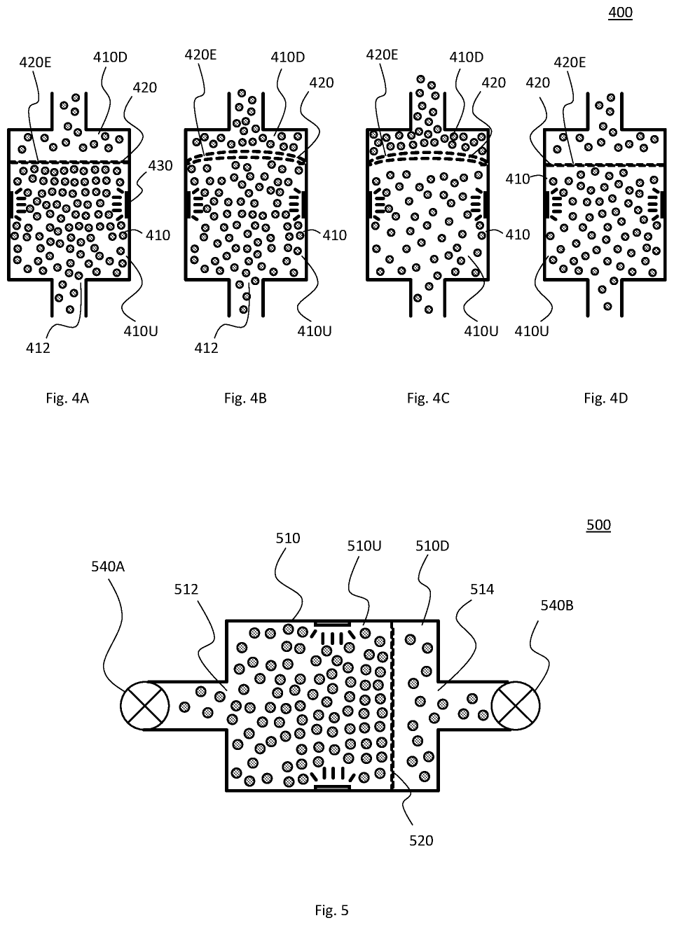

FIG. 4A-FIG. 4D show schematic diagrams of different stages of an exemplary embodiment of the sanitizing apparatus 400. Sanitizing apparatus 400 has similar features as the sanitizing apparatus 100 in FIG. 1. The same features have the same last two digits and letter (if any) in their reference numbers. Referring to FIG. 4A, the divider 420 may include at least one opening 420E and is flexible, such that the divider 420 may be adapted to flex between an original position (as shown in FIG. 4A and a flexed position (as shown in FIG. 4B), such that in the original position, the at least one opening 420E is closed to prevent the fluid therethrough and in the flexed position, the at least one opening 420E is opened to allow the fluid therethrough. As shown in FIG. 4A, the divider 420 may include a plurality of openings 420E. Referring to FIG. 4A, the divider 420 may be adapted to obstruct the flow of the fluid in the upstream portion 410U of the chamber 410. As the fluid is being pumped into the upstream portion 410U of the chamber 410, it is retained and agglomerated therein. Gradually, the fluid density and fluid pressure in the upstream portion 410U increases. At the same time, the fluid in the upstream portion 410U is being sanitized by the sanitizing light emitted from the sanitizing light source 430. Divider 420 may be flexed by the fluid in the upstream chamber 410 from the original position towards the flexed position as shown in FIG. 4B, such that in the flexed position, the plurality of openings 420E are opened sufficiently for the fluid to pass through. Hence, the fluid may be released from the upstream portion 410U of chamber 410.

As shown in FIG. 4C, the pressure will continue to increase until the divider 420 is in the flexed position where the plurality of openings 420E are opened to allow the fluid to escape therethrough into the downstream portion 410D of the chamber 410. Divider 420 may be elastic such that when the fluid pressure in the upstream portion 410U of the chamber 410 decreases, the divider 420 may retract from the flexed position as shown in FIG. 4C to the original position as shown in FIG. 4D. In the original position, the plurality of openings 420E may be closed, and the fluid is trapped and obstructed in the upstream portion 410U of the chamber 410. As shown in FIG. 4D, the fluid may continue to be directed into the chamber 410 and be accumulated until the fluid pressure within the upstream portion 410U reaches a level where the divider 420 is being pushed to the flexed position where the fluid escapes again. Divider 420 may continue to flex from the original position to the flexed position and back as the fluid is being directed into the chamber 410. In this way, it is possible to retain the fluid in the upstream portion 410U of the chamber 410 sufficiently long enough for the fluid to be completely sanitized. The flexibility and/or elasticity of the divider 420 may be chosen based on the fluid pressure required and/or the time required for the sanitization. Divider 420 may include a diaphragm, e.g. an elastic diaphragm, a permeable/semi-permeable membrane.

FIG. 5 shows a schematic diagram of an exemplary embodiment of the sanitizing apparatus 500. Sanitizing apparatus 500 has similar features as the sanitizing apparatus 100 in FIG. 1. The same features have the same last two digits and letter (if any) in their reference numbers. Sanitizing apparatus 500 may include an inlet pump 540A adapted to pump fluid into the chamber 510 via the inlet 512. While it is possible to connect the sanitizing apparatus 500 to a pressurized fluid source, the sanitizing apparatus 500 having the inlet pump 540A may allow it to be portable. Sanitizing apparatus 500 may include an outlet pump 540B adapted to draw fluid out of the chamber 510 via the outlet 514. Instead of increasing the fluid pressure in the upstream portion 510U of the chamber 510 via the inlet 512, it is possible to draw the fluid out from the downstream portion 510D of the chamber 510 to create a vacuum so that the divider 520 may be flexed from the original position to the flexed position as shown in FIG. 4. Sanitizing apparatus 500 may include an inlet valve (not shown in FIG. 5) to control the fluid flow into the chamber 510 and/or an outlet valve (not shown in FIG. 5) to control the fluid flow out of the chamber 510. Inlet valve and outlet valve may be check valves. Both inlet valve and outlet valve may be controlled to control the pressure within the upstream portion 510U and downstream portion 510D of the chamber 510 respectively.

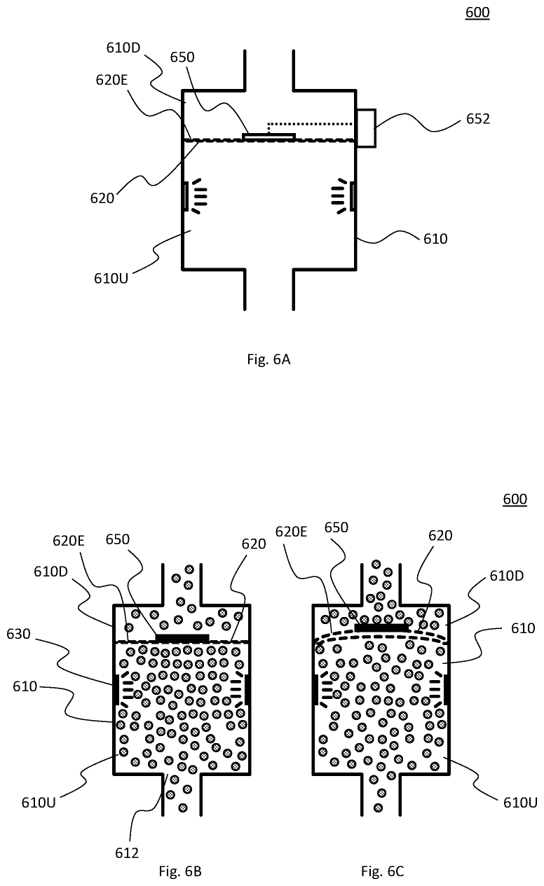

FIG. 6A-FIG. 6C show schematic diagrams of different stages of an exemplary embodiment of the sanitizing apparatus 600. Sanitizing apparatus 600 has similar features as the sanitizing apparatus 100 in FIG. 1. The same features have the same last two digits and letter (if any) in their reference numbers. As shown in FIG. 6A, the sanitizing apparatus 600 may include an actuator 650 adapted to actuate the divider 620 between the original position (as shown in FIG. 6B) and the flexed position (as shown in FIG. 6C), such that in the flexed position, the at least one opening 620E is opened for the fluid to flow therethrough and be released from the upstream portion 610U to the downstream portion 610D of the chamber 610. Actuator 650 may be attached to the divider 620 at its centre such that the divider 620 may be actuated from its centre. Actuator 650 may be attached to the divider 620 at the side facing the downstream portion 610D of the chamber 610. Referring to FIG. 6A, the sanitizing apparatus 600 may include a controller 652 configured to control the actuator 650. Divider 620 may have a plurality of openings 620E. Actuator 650 may include a piezoelectric actuator, a motorised actuator, a pneumatic actuator, etc. Actuator 650 may be connected to the chamber wall and retracted to flex the divider 620.

Referring to FIG. 6B, as the fluid is directed into the upstream portion 610U of the chamber 610 via the inlet 612, the fluid is being accumulated and compressed therein. Actuator 650 may be actuated to flex the divider 620 from the original position to the flexed position as shown in FIG. 6C. In the original position, the plurality of openings 620E may be closed, and the divider 620 may retain the compressed fluid in the upstream portion 610U of the chamber 610. When the fluid is in the upstream portion 610U, the fluid may be sanitized by the sanitizing light from the sanitizing light source 630. When in the flexed position, the plurality of openings 620E may be opened, and the fluid may pass therethrough. Hence, the fluid may be released from the upstream portion 610U to the downstream portion 610D of the chamber 610. Once the fluid is released from the upstream portion 610U, the actuator 650 may be actuated to return the divider 620 from the flexed position to the original position such that the plurality of openings 620E in the divider 620 may be closed. When the fluid is released from the upstream portion 610U, the fluid pressure in the upstream portion 610U may be reduced. Divider 620 may be elastic such that, when the fluid pressure is reduced, the divider 620 may retract from the flexed position to the original position without being actuated by the actuator 650. Divider 620 may continue to be flexed from the original position to the flexed position and back during the sanitization of the fluid through the chamber 610.

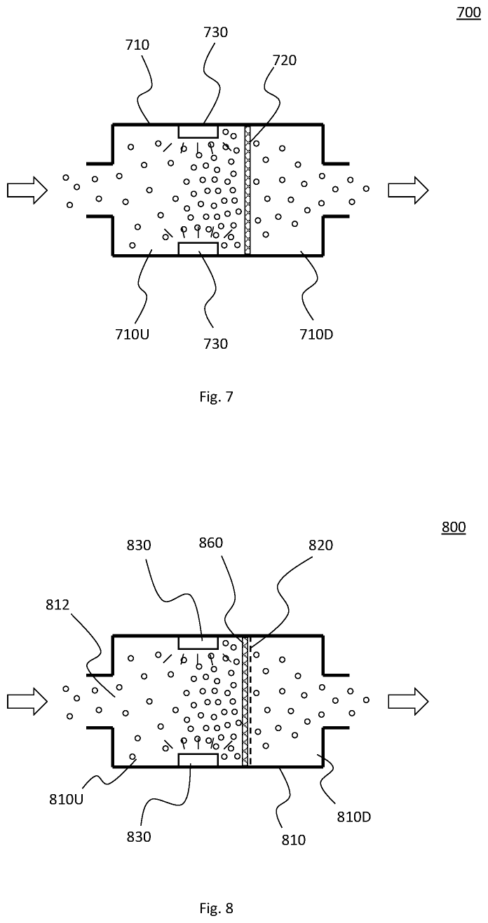

FIG. 7 shows a schematic diagram of an exemplary embodiment of the sanitizing apparatus 700. Sanitizing apparatus 700 has similar features as the sanitizing apparatus 100 in FIG. 1. The same features have the same last two digits and letter (if any) in their reference numbers. As shown in FIG. 7, the divider 720 may include a plurality of micropores adapted to allow the fluid therethrough. Plurality of micropores are adapted to restrict the fluid flow therethrough. As the fluid is being directed into the upstream portion 710U of the chamber 710, the fluid may be obstructed or restricted by the divider 720. Due to the micropores, the flowrate of the fluid across the divider 720 from the upstream portion 710U to the downstream portion 710D of the chamber 710 may be substantially reduced. As the fluid is being restricted by the divider 720, the fluid may be compressed in the upstream portion 710U and density of the fluid is increased. Fluid may then be sanitized by the sanitizing light from the sanitizing light source 730. In this way, the duration of exposure by the sanitizing light and the amount of fluid being sanitized are increased. Divider 720 may include a fibre optic mesh having the plurality of micropores and adapted to transmit and emit the sanitizing light onto the fluid therethrough. As the fluid is being obstructed by the divider 720, besides the sanitizing light from the sanitizing light source 730, the fluid may be sanitized by the sanitizing light from the divider 720. Further, as the fluid flows through the divider 720 from the upstream portion 710U to the downstream portion 710D, the fluid may be sanitized by the sanitizing light from the divider 720. In this way, the duration of exposure of the fluid by the sanitizing light is increased substantially compared to conventional fluid sanitizer.

FIG. 8 shows a schematic diagram of an exemplary embodiment of the sanitizing apparatus 800. Sanitizing apparatus 800 has similar features as the sanitizing apparatus 100 in FIG. 1. The same features have the same last two digits and letter (if any) in their reference numbers. In addition to the divider 820, the sanitizing apparatus 800 may include a restrictor 860 adapted to restrict the fluid flow from the inlet 812 therethrough. Restrictor 860 may extend across the chamber 810 to divide the upstream portion 810U of the chamber 810 into two sections. Restrictor 860 may be disposed between the divider 820 and the inlet 812, i.e. upstream of the divider 820, and adapted to reduce the flowrate of the fluid therethrough. In this way, the density of the fluid before the restrictor 860 may be increased. As shown in FIG. 8, the restrictor 860 may be disposed downstream of the sanitizing light source 830 so that the fluid restricted by the restrictor 860 may be sanitized by the sanitizing light. After the fluid flows through the restrictor 860, the fluid may be obstructed by the divider 820 as mentioned in any one of the above embodiments. Sanitizing apparatus 800 may include sanitizing light source 830 (not shown in FIG. 8) between the restrictor 860 and the divider 820 to increase the exposure duration of the fluid to the sanitizing light. Restrictor 860 may include a fibre optic mesh having a plurality of micropores and adapted to transmit and emit the sanitizing light onto the fluid therethrough. As the fluid is being restricted by the restrictor 860, besides the sanitizing light from the sanitizing light source 830, the fluid may be sanitized by the sanitizing light from the restrictor 860. Further, as the fluid flows through the restrictor 860, the fluid may be sanitized by the sanitizing light from the restrictor 860.

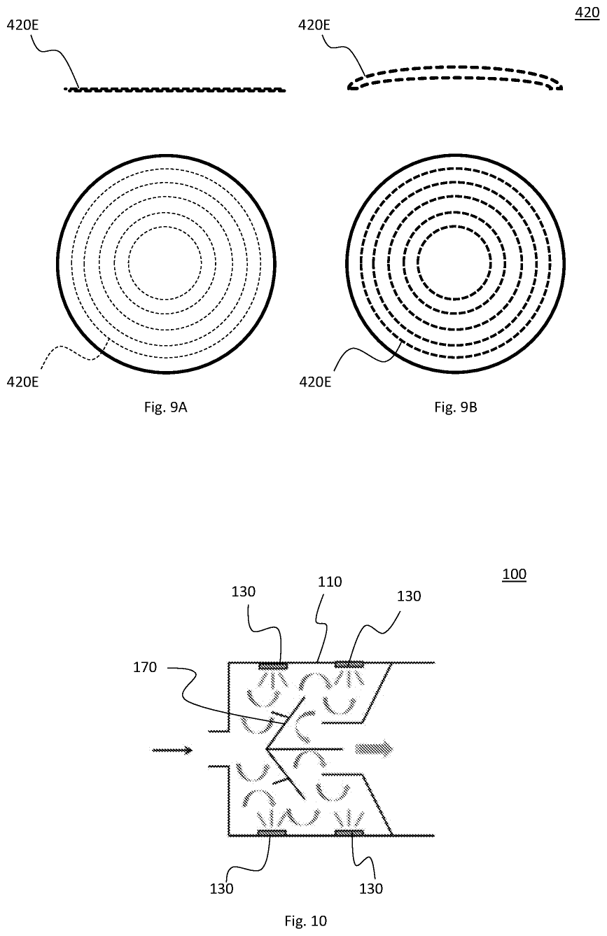

FIG. 9A and FIG. 9B show top and elevation views of an exemplary embodiment of the divider 420 as shown in one of the embodiments in FIG. 4-FIG. 6. As shown in FIG. 9A, the divider 420 may include a plurality of openings 420E. Plurality of openings 420E may be arranged in a concentric arrangement. In FIG. 9A, the divider 420 may be in the original position where the plurality of openings 420E are in the closed position. In FIG. 9B, the divider 420 may be in the flexed position where the plurality of openings 420E are in the opened position. Plurality of openings 420E may be formed by cutting a plurality of slits in the divider 420. In the closed position, the slits may be closed or narrow enough to prevent fluid from going therethrough. As the divider 420 may be elastic, the slits are expanded into gaps when the divider 420 is stretched or flexed outwardly. When expanded sufficiently, the fluid may be able to flow through the gaps. When the divider 420 is retracted, the gaps may contract back to slits hence preventing the fluid from flowing therethrough. Actuator 650 (not shown in FIG. 9) may be connected to the divider 420 at its centre portion so that the divider 420 may be actuated from its centre to allow the plurality of openings 420E to open evenly throughout the divider 420 when it is flexed or expanded. While it is shown that the divider 420 may be circular, the divider 420 may be in other shapes, e.g. square, hexagonal, etc. Consequently, the arrangement of the plurality of openings 420E may be arranged according to the shape of the divider 420. Divider 420 may be made from a material with micro or nano-sized pores that actively restricts fluid flow, thus allowing a pressure build up and fluid compression before the fluid have enough energy to penetrate the material. Divider 420 may include permeable, semi-permeable, or porous membrane. Divider 420 may be rigid or flexible.

FIG. 10 shows a schematic diagram of a plurality of deflectors 170 adapted to be used in the sanitizing apparatus 100 or any one of the above embodiments of the sanitizing apparatus. Sanitizing apparatus 100 may include a plurality of deflectors 170 adapted to deflect the fluid flow direction in the chamber 110. A plurality of deflectors 170 may be transparent to allow sanitizing light to pass through to increase the exposure of the fluid to the sanitizing light. Plurality of deflectors 170 may be reflective to reflect the sanitizing light from the sanitizing light source 130 to the fluid. As shown in FIG. 10, the plurality of deflectors 170 may be adapted to channel the fluid to flow in a longer flow path to increase the duration of exposure of the fluid to the sanitizing light. Deflector may be adapted to create turbulence flow in the chamber 110 so as to increase the exposure of the fluid to the sanitizing light. Plurality of deflectors 170 may have a rough surface to increase adhesion of the fluid to the surface to reduce the flow rate of the fluid along them. The rough surface may create a multilayer local compaction of air. When sanitizing air, the air pressure and obstruction in the chamber 110 may cause air molecule adsorption on the surface of the deflector to create multilayer local compaction of the air on the surface.

Sanitizing light source 130 may include UV LED adapted to emit UV light. UV LED light source may include UVC light source for emitting UVC light. Sanitizing light source 130 may include a UV LED having a power of more than 20 mW at a wavelength range of 265 nm to 300 nm.

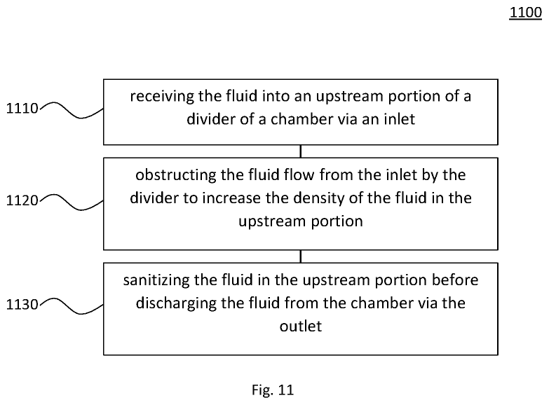

FIG. 11 shows an exemplary sanitizing method 1100 for sanitizing a fluid. Method 1100 includes receiving the fluid into an upstream portion 110U of a divider 120 of a chamber 110 via an inlet 112 in step 1110, obstructing the fluid flow from the inlet 112 by the divider 120 to increase the density of the fluid in the upstream portion 110U in step 1120, and sanitizing the fluid in the upstream portion 110U before discharging the fluid from the chamber 110 via the outlet 114 in step 1130.

A skilled person would appreciate that the features described in one example may not be restricted to that example and may be combined with any one of the other examples.

The present invention relates to a sanitizing apparatus for sanitizing a fluid and a sanitizing method thereof generally as herein described, with reference to and/or illustrated in the accompanying drawings.

1-21. (canceled)

22. A portable sanitizing apparatus for sanitizing a fluid, the portable sanitizing apparatus comprising:
a chamber comprising an inlet for receiving the fluid thereinto and an outlet for discharging the fluid therefrom;
a divider comprising a diaphragm disposed within the chamber and between the inlet and the outlet, the diaphragm extends across the chamber and adapted to divide the chamber into an upstream portion in fluid communication with the inlet and a downstream portion in fluid communication with the outlet, wherein the diaphragm comprises at least one opening and is flexible, wherein the diaphragm is adapted to flex between an original position and a flexed position, wherein in the original position, the at least one opening is closed to prevent the fluid therethrough and in the flexed position, the at least one opening is opened to allow the fluid therethrough; and
a sanitizing light source disposed in the upstream portion and adapted to emit a sanitizing light to sanitize the fluid therein,
wherein the diaphragm is adapted to obstruct a fluid flow from the inlet to increase a density of the fluid in the upstream portion, wherein the fluid is sanitized and is adapted to be flexed by the fluid in the upstream portion from the original position to the flexed position, wherein in the flexed position, the at least one opening is opened for the fluid to be released from the upstream portion to the downstream portion, wherein the diaphragm is adapted to retain the fluid from the inlet in the upstream portion to be sanitized and release the fluid into the downstream portion to be discharged from the chamber via the outlet.

23. The portable sanitizing apparatus according to claim 1, further comprising an actuator adapted to actuate the diaphragm between the original position and the flexed position, wherein in the flexed position, the at least one opening is opened for the fluid to be released from the upstream portion.

24. The portable sanitizing apparatus according to claim 1, wherein the divider comprises a plurality of micropores adapted to allow the fluid therethrough.

25. The portable sanitizing apparatus according to claim 3, wherein the divider comprises a fibre optic mesh comprising the plurality of micropores and adapted to transmit and emit the sanitizing light onto the fluid therethrough.

26. The portable sanitizing apparatus according to claim 1, further comprising a restrictor adapted to restrict the fluid flow from the inlet therethrough.

27. The portable sanitizing apparatus according to claim 5, wherein the restrictor comprises a fibre optic mesh comprising a plurality of micro openings and adapted to transmit and emit the sanitizing light onto the fluid therethrough.

28. The portable sanitizing apparatus according to claim 1, wherein the divider comprises a rough surface facing the inlet, wherein the rough surface is adapted to reduce the fluid flow rate along the divider.

29. The portable sanitizing apparatus according to claim 1, further comprising an inlet pump adapted to pump fluid into the chamber via the inlet.

30. The portable sanitizing apparatus according to claim 1, further comprising an outlet pump adapted to draw fluid out of the chamber via the outlet.

31. A sanitizing method for sanitizing a fluid in a portable sanitizing apparatus comprising a chamber comprising an inlet for receiving the fluid thereinto and an outlet for discharging the fluid therefrom and a divider comprising a diaphragm disposed between the inlet and the outlet, the method comprising:
receiving the fluid into an upstream portion of the diaphragm in the chamber via the inlet;
obstructing a fluid flow from the inlet by the diaphragm to increase a density of the fluid in the upstream portion;
sanitizing the fluid in the upstream portion before discharging the fluid from the chamber via the outlet; and
flexing the diaphragm between an original position and a flexed position, wherein in the original position, at least one opening of the diaphragm is closed to prevent the fluid therethrough and in the flexed position, the at least one opening is opened to allow the fluid therethrough, wherein the diaphragm is adapted to be flexed by the fluid in the upstream portion from the original position to the flexed position, wherein in the flexed position, the at least one opening is opened for the fluid to be released from the upstream portion, wherein the fluid from the inlet is retained in the upstream portion to be sanitized and released into a downstream portion of the diaphragm to be discharged from the chamber via the outlet.

32. The sanitizing method according to claim 10, further comprising actuating the diaphragm between the original position and the flexed position, wherein in the flexed position, the at least one opening is opened for the fluid to be released from the upstream portion.

33. The sanitizing method according to claim 10, further comprising pumping the fluid into chamber via the inlet.

34. The sanitizing method according to claim 10, further comprising drawing the fluid out of the chamber via the outlet.
